(19) United States
(12) Patent Application Publication
(10) Pub. No.: US 2013/0289499 A1
(43) Pub. Date: Oct. 31, 2013

(54) HAIR LOSS PREVENTION COMPOSITION AND METHOD

(71) Applicant: Yulia Zakidaiskaia, Foster City, CA (US)

(72) Inventor: Yulia Zakidaiskaia, Foster City, CA (US)

(21) Appl. No.: 13/869,933

(22) Filed: Apr. 24, 2013

Related U.S. Application Data
(60) Provisional application No. 61/639,084, filed on Apr. 27, 2012.

(51) Int. Cl. A61K 33/14

(52) U.S. Cl. CPC A61K 33/14
USPC 604/290; 424/680

(57) ABSTRACT

A composition of matter for preventing hair loss. An aqueous solution of sodium chloride and water in the range of 30-35.9 parts sodium chloride per 100 ml of water, applied to the scalp in a determined manner to prevent loss of hair. Hair on a scalp is portioned prior to application of the composition of matter. The scalp is massaged, and a polymeric cover positions over the scalp. A warmth generating cover positions over the polymeric cover to provide additional warmth on the scalp.

### CLAIM OF PRIORITY

This application claims priority from U.S. Provisional Patent 61/639084 filed Apr. 27, 2012, the entirety of the specification of which is hereby incorporated by reference as if fully set forth in the present specification.

### BACKGROUND OF THE INVENTION

The present invention relates generally to compositions and more specifically to compositions for preventing hair loss.

Hair loss or alopecia might be caused by any number of reasons. As an example, chemotherapy used for eradicating cancer cells in patients can result in hair loss, particularly in women. Thus, although chemotherapy is beneficial for destroying rapidly-dividing cancer cells, it has the unintended side effect of damaging a patient's hair follicles.

Another reason for hair loss may relate to hormones related to pregnancy and/or lactation. Women typically experience hair loss during or after lactation. Further yet, hair loss might simply be a byproduct of old age, in which case, hair follicles begin to shrink and die off.

One common solution to hair loss is Minoxidil, otherwise known as Rogaine. Minoxidil revitalizes shrunken hair follicles by increasing blood flow into the hair follicles. That is, Minoxidil is a vasodilator. Among other disadvantages, Minoxidil works for some patients and is ineffective for others. Further yet, Minoxidil cannot be discontinued, otherwise, hair loss begins to reoccur.

Typically, increasing blood flow in the scalp through massaging, laser lights, and chemicals has been used to help increase hair growth. These techniques have had limited, if any, success.

There is a need to address one or more of the foregoing disadvantages of conventional systems and methods, and the present invention meets this need.

### BRIEF SUMMARY OF THE INVENTION

Various aspects of a hair prevention composition and method can be found in exemplary embodiments of the present invention. In one embodiment, a composition of matter comprises an aqueous solution of sodium chloride and water in the range of 30-35.9 parts sodium chloride per 100 ml of water, applied to the scalp in a determined manner for a predetermined duration to prevent loss of hair.

However, in other embodiments, the composition may incorporate different ionic salts with blood vessel dilating properties and/or an eclectic assortment of ingredients efficacious for inhibiting hair loss. Results from inventor experimentation and analysis determined that a proper amount and application method of NaCl, sodium chloride, can dilate blood vessels in the skin of the scalp, increasing blood flow to the scalp to strengthen hair follicles and combat hair loss.

The method includes an initial step of partitioning the hair in sections. Partitioning the hair allow a user to regulate the application of the composition to desired areas of the hair. After partitioning, the composition is applied with a dispenser, which is configured to preserve the integrity of the composition. Those skilled in the art, in light of the present teachings, will recognize that different durations of saturation can provide the desired therapeutic effects.

Once all of the segments and the entirety of the scalp have received the aqueous solution, a polymeric cover, such as a shower cap, is at least partially placed over the head. The polymeric cover initiates the process of retaining warmth between the scalp and the cover. The polymeric cover can be grafted to include various polymers, oligomers, and small moieties to regulate the amount of warmth generated on the scalp.

In some embodiments, a warmth generating cover at least partially covers the polymeric cover to provide additional thermal energy in proximity to the scalp. After the warmth generating cover is applied, the aqueous solution is allowed to remain on the head for about 20 to 30 minutes after which point the hair can then be washed, shampooed or otherwise cleaned to remove the aqueous solution from the hair.

A further understanding of the nature and advantages of the present invention herein may be realized by reference to the remaining portions of the specifications and the attached drawings. Further features and advantages of the present invention as well as the structure and operation of various embodiments of the present invention are described in detail below with respect to the accompanying drawings. In the drawings, the same reference numbers indicate identical or functionally similar elements.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates a dispenser according to an exemplary embodiment of the present invention;

FIG. 2 illustrates a polymeric cover according to an exemplary embodiment of the present invention;

FIG. 3 illustrates a warmth generating cover according an exemplary embodiment of the present invention; and

FIG. 4 illustrates a method for using a 30-35.9% aqueous solution according to an exemplary embodiment of the present invention.

### DETAILED DESCRIPTION OF THE INVENTION

Reference will now be made in detail to the embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth to provide a thorough understanding of the present invention. However, it will be obvious to one of ordinary skill in the art that the present invention may be practiced without these specific details. In other instances, well-known methods, procedures, components, and circuits have not been described in detail so as to not unnecessarily obscure aspects of the present invention.

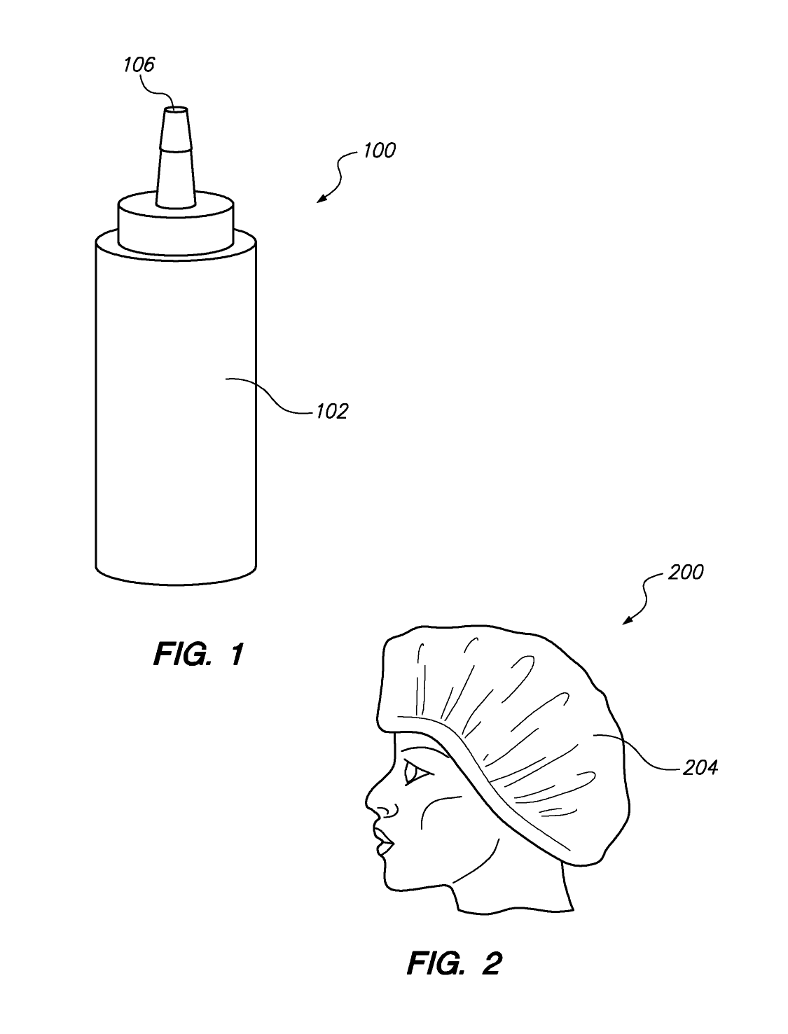

FIG. 1 illustrates dispenser 100 according to an exemplary embodiment of the present invention.

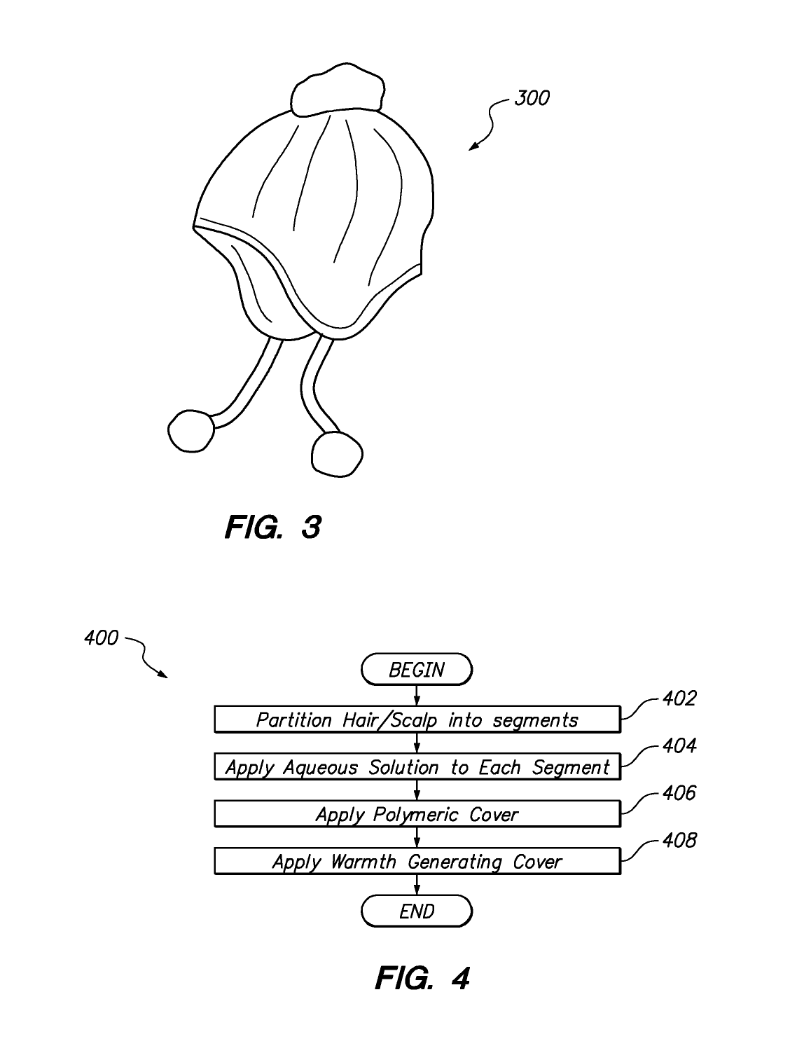

In FIG. 1, a dispenser 100 comprises a body portion 102 for storing composition of matter herein disclosed according to the principles of the present invention and further discussed below with reference to FIGS. 3-4. Dispenser 100 is preferably airtight and prevents air from crystallizing the composition of the present invention.

In yet another embodiment, dispenser 100 may include an applicator 106 configured to engage the scalp and efficiently apply the aqueous solution. Applicator 106 may include, without limitation, a flat surface, brushes, stimulating protrusions, dispensing velocity regulators, shut off valve, and a handle. Body portion 102 may further include a rubber grip for transporting and controlling dispenser 100 during application of the composition.

FIG. 2 illustrates polymeric cover 204 according to an exemplary embodiment of the present invention.

In FIG. 2, as shown, user 202 wears polymeric cover 202 over the head after application of the present composition of matter. Polymeric cover 202 is sufficiently flexible to at least partially cover a head.

The physical properties of polymeric cover 202 are efficacious for retaining heat in proximity to the scalp, partially due to the absence of pores and ventilation in the polymer material. In some embodiments, polymeric cover 202 can be grafted to include various polymers, oligomers, and small moieties for initiating different amounts of thermal energy on the scalp. Grafting additional polymers on a surface of the polymeric cover 202 may further reduce ventilation, thereby increasing the retention of warmth on the scalp.

In one embodiment, the composition of matter of the present invention comprises an aqueous solution of sodium chloride. Specifically, predetermined amounts of sodium chloride salts are dissolved in predetermined amounts of water. When dissolved in water, the sodium chloride molecule dissolves into positive ions Sodium Na⁺ (Natrium) and negative Cl⁻ ions.

NaCl(s)=Na(aq)+Cl⁻(aq)

When NaCl dissolves in water, the salt disassociates rapidly so that the conductivity of the solution, therefore, increases rapidly. Consequently, the present inventor having conducted various tests, applying aqueous NaCl to the scalp, has determined that aqueous NaCl exhibits blood vessel dilator properties that prevents or retards hair loss particularly in the critical range 30-35.9% concentration of NaCl.

Specifically, aqueous sodium chloride acts to dilate blood vessels to increase blood flow to shrunken particles and supply essential nutrients and oxygen to hair root cells thus resulting in revived and strengthened hair follicles that support healthy hair growth and stops hair fall out. Specifically, aqueous sodium chloride acts to dilate blood vessels in skin of the scalp to increase blood flow to shrunken and dormant hair follicles and increases the supply of essential nutrients and oxygen to hair root cells.

In one embodiment of the present invention, an aqueous solution of 30% sodium chloride volume weight is produced. That is, 30 mg of NaCl is added to 100 ml of H2O (water). This 30 percent aqueous solution when applied to the scalp in accordance with the method of the present invention prevents the occurrence of hair loss as will be further described with reference to FIG. 4.

Those skilled in the art will recognize that the quantity of NaCl may be varied to adapt to different scalps. For example, without limitation, a dry scalp may necessitate a smaller amount of NaCl solution, which forms a more hypotonic solution. The lesser NaCl solutes found in the hypotonic solution allow additional water to migrate towards pores in the dry scalp, thereby increasing hydration.

In one embodiment of the present invention, the solution may include any ionic salts with blood vessel dilating properties.

Additional ingredients that can be added to the composition include, without limitation, a thickening agent, an odor enhancing agent, a coloring agent, and an antibacterial agent.

In one alternative embodiment, the solution consists of a NaCl concentration of 35% and 2% Xanthan Gum that serves as a thickening agent. This composition forms a thin gel that facilitates application on the scalp.

FIG. 3 illustrates warmth generating cover 300 according an exemplary embodiment of the present invention.

Warmth generating cover 300 is preferably made of wool or other comparable materials. Warmth generating cover 300 might be a beanie or the like used by user 302 (FIG. 3) over the polymeric cover 202 after application of the composition of matter.

Warmth generating cover 300 contours shape of polymeric cover 202, serving to provide an additional barrier for retaining warmth in proximity to the head. The additional warmth serves as a catalyst that expedites and enhances the hair prevention properties of the composition. In some embodiments, warmth generating cover 300 helps retain additional moisture on the scalp by inhibiting evaporation.

Warmth generating cover 300 comprises an inner surface that engages polymeric cover 202. Inner surface comprises a smooth material, such as plastic, for facilitated adornment and removal of warmth generating cover 300.

FIG. 4 illustrates method 400 for using a 30% aqueous solution according to an exemplary embodiment of the present invention.

In FIG. 4, block 402 includes user 202 (FIG. 2) partitioning the hair or scalp into segments. Partitioning the hair into smaller segments makes it easier to apply the aqueous solution in a stepwise fashion until the entirety of the scalp is completely and adequately covered. Note that the user may or may not choose to use a band (not shown) around the forehead to prevent the aqueous solution from dripping onto the face.

At block 404, user 202 begins to apply the aqueous solution to each of the aforementioned segments of the scalp. Here, the aqueous solution is contained within dispenser 100. Specifically, user 202 inverts dispenser 100 to apply the solution onto the scalp via applicator 106. Once a segment is completed, user 202 moves to the next segment and repeats the process by applying the aqueous solution to that particular segment until all scalp partitions are covered with applications of aqueous NaCl solution. After each application, the solution is rubbed into the scalp.

As the aqueous solution is applied, user 202 begins to feel either a tingling sensation and/or warmth on the scalp. User 202 may experience warmth and a tingling sensation. The degree of warmth is different for everyone. Some experience pleasant warmth while others may experience a more burning sensation. In one embodiment, aqueous solution is applied while simultaneously partitioning the hair.

At block 406, once all of the segments and the entirety of the scalp have received the aqueous solution, polymeric cover 204, such as a shower cap, is placed over the head. Polymeric cover 204 initiates the process of generating some warmth over the scalp. Polymeric cover 204 is configured to cover the whole scalp, or a limited area of the scalp. In some embodiments, fasteners secure polymeric cover 202 to the scalp.

After the polymeric cover 204 is applied at step 408, warmth-generating cover 300 positions over polymeric cover 204. As implied by its name, warmth-generating cover 300 generates additional warmth around the scalp in addition to polymeric cover 204.

After the warmth-generating cover 300 is applied, the aqueous solution is allowed to remain on the head for about 20 to 30 minutes after which point the hair can then be washed, shampooed or otherwise cleaned to remove the aqueous solution from the hair.

In one embodiment, application of the aqueous solution to the scalp is followed up by massaging the scalp and applying warmth generating cover 300 on the polymeric cover for at least fifteen minutes. This process must be repeated once a week for at least one month to see desired results. Those skilled in the art will recognize that different scalps will experience different levels of hair loss prevention, and in different time scales.

An advantage of the present invention is that it uses an aqueous solution that is relatively inexpensive and can be applied by consumers without any special expertise unlike prior art that employs difficult to find solutions such as Aloe Vera and the like that must be processed and manufactured. Another advantage of the present invention is that unlike Minoxidil, for example, the use of the present invention can be discontinued as desired or used PRN (as needed) as desired by user 202.

Experimentation was conducted using various concentrations, the following concentrations being found to be optimum.

TABLE 1
Female, Age 59

| | | Warmth or | | | |
|---|---|---|---|---|---|
| Hair | Concen- | Tingling | Frequency | Du- | |
| Loss | tration | Sensation | of Use | ration | Result |
| Older | 30% | Medium | Every two | 2 | Hair loss stoppage, |
| age | | | days | months | some hair growth |

In Table 1, a 30 percent concentration was applied to an adult, age 59. Hair loss cause is old age. The warmth or tingling sensation was medium; frequency of use was every two days; the duration of use was two months, and there was some hair loss stoppage and some hair growth.

TABLE 2
Female, Age 33

| | | Warmth or | | | |
|---|---|---|---|---|---|
| Hair | Concen- | Tingling | Frequency | Du- | |
| Loss | tration | Sensation | of Use | ration | Result |
| Stress, | 35% | Maximum | One time | 2 | Hair loss |
| lack of | | | every | months | decreased |
| sleep, | | | week, 10 | | to a minimum, |
| vitamin | | | minutes | | new “baby |
| deficiency | | | per appli- | | hair” growth |
| | | | cation | | appeared |

In Table 2, a 35 percent concentration was applied to an adult, age 33. Hair loss cause is stress, lack of sleep, and vitamin deficiency. The warmth or tingling sensation was maximum; frequency of use was one time every week, 10 minutes per application; the duration of use was two months. Hair loss decreased to a minimum, new “baby hair” growth appeared.

TABLE 3
Male, Age 31

| | | Warmth or | | | |
|---|---|---|---|---|---|
| Hair | Concen- | Tingling | Frequency | Du- | |
| Loss | tration | Sensation | of Use | ration | Result |
| Receding | 35% | Maximum | 3 times a | 3 | New hair growth |
| hairline | | | week, 10 | months | in the hairline |
| along the | | | minutes | | and throughout |
| top of the | | | per appli- | | the head occur- |
| forehead | | | cation | | red, the user |
| | | | | | also noticed |
| | | | | | strands of hair |
| | | | | | growing from |
| | | | | | the same follicles |

In Table 3, a 35 percent concentration was applied to an adult, age 31. Hair loss cause is receding hairline along the top of the forehead. The warmth or tingling sensation was maximum; frequency of use was 3 times a week, 10 minutes per application; the duration of use was three months.

TABLE 4
Female, Age 62

| | | Warmth or | | | |
|---|---|---|---|---|---|
| Hair | Concen- | Tingling | Frequency | Du- | |
| Loss | tration | Sensation | of Use | ration | Result |
| chemo- | 35% | Medium | Every two | 2 | hair loss |
| therapy | | | days | months | stoppage; |
| | | | | | re-growth |
| | | | | | to cover |
| | | | | | bald spot |

In Table 4, a 35 percent concentration was applied to an adult, age 62. Hair loss cause is chemotherapy. The warmth or tingling sensation was medium; frequency of use was three times per week, 15 minutes per application; the duration of use was two months, and there was hair loss stoppage and some hair growth along a bold spot in the upper part of the head—the size of a fist. The result was new hair growth resulting in the bold spot disappearance.

TABLE 5
Female, Age 73

| | | Warmth or | | | |
|---|---|---|---|---|---|
| Hair | Concen- | Tingling | Frequency | Du- | |
| Loss | tration | Sensation | of Use | ration | Result |
| chemo- | 30% | Medium | 1 time per | 2 | Hair loss |
| therapy | | | week, 15 | months | decreased to a |
| | | | minutes | | minimum, new |
| | | | per appli- | | hair growth |
| | | | cation | | occurred. The |
| | | | | | user had gray |
| | | | | | hair due to |
| | | | | | her age, but |
| | | | | | noticed new |
| | | | | | hair in its |
| | | | | | original brown |
| | | | | | color |

In Table 5, 30 percent concentration was applied to an adult, age 73. Hair loss cause is chemotherapy. The warmth or tingling sensation was medium; frequency of use was 1 time per week, 15 minutes per application; the duration of use was two months. Hair loss decreased to a minimum, new hair growth occurred. The user had gray hair due to her age, but noticed a phenomenon that new hair grew out in its original brown color.

TABLE 6
Female, Age 37

| | | Warmth or | | | |
|---|---|---|---|---|---|
| Hair | Concen- | Tingling | Frequency | Du- | |
| Loss | tration | Sensation | of Use | ration | Result |
| child- | 35.9% | Maximum | 2 times per | 2 | hair loss |
| birth, | | | week, 20 | months | decreased |
| lac- | | | minutes | | to a minimum, |
| tation | | | per appli- | | new “baby hair” |
| | | | cation | | growth occurred |

In Table 6, a 35.9 percent concentration was applied to an adult, age 37. Hair loss cause is childbirth and lactation. The warmth or tingling sensation was maximum; frequency of use was 2 times per week, 20 minutes per application; the duration of use was two months and hair loss decreased to a minimum, new “baby hair” growth occurred. Note that the criticality of the range 30%-35.9% concentration by weight of sodium chloride aqueous solution is demonstrated by the above-referenced experimentation which illustrate maximum efficiency within this range.

While the above is a complete description of exemplary specific embodiments of the invention, additional embodiments are also possible. For example, in an alternate embodiment, the present invention is not limited to NaCl. Although not shown, any ionic salt consistent with the spirit and scope of the present invention can be utilized. Thus, the above description should not be taken as limiting the scope of the invention, which is defined by the appended claims along with their full scope of equivalents.

I claim:

1. A composition of matter for preventing hair loss, said composition comprising:
an aqueous solution of sodium chloride and water, said sodium chloride having a concentration in the range of 30-35.9 parts sodium chloride per 100 ml of water, wherein upon applying said aqueous solution to a scalp of a human being for designated periods, said solution prevents loss of hair.

2. The composition of claim 1, wherein said aqueous solution comprises a thickening agent.

3. The composition of claim 2, wherein said thickening agent consists of about 2% Xanthan Gum.

4. The composition of claim 1, wherein said polymeric cover comprises a shower cap.

5. The composition of claim 1, wherein said warmth generating cover comprises a wool cap.

6. The composition of claim 1, wherein said composition of matter is configured to be applied on said scalp from a dispenser.

7. A method operable to apply a composition of matter for preventing hair loss, said method comprising:
partitioning hair on a scalp into segments;
applying an aqueous solution to said segments with a dispenser;
applying a polymeric cover to at least partially cover said scalp; and
applying a warmth generating cover to at least partially cover said polymeric cover.

8. The method of claim 7 wherein partitioning hair comprises combing hair into segments so as to expose the scalp for application of said aqueous solution.

9. The method of claim 7 wherein applying said aqueous solution comprises a dispenser.

10. The method of claim 7 wherein applying said aqueous solution comprises massaging said scalp.

11. The method of claim 7 wherein applying a polymeric cover comprises a shower cap.

12. The method of claim 7 wherein applying a warmth generating cover comprises retaining said warmth generating cover on said polymeric cover for at least 15 minutes.

13. A composition of matter for preventing hair loss, said composition comprising:
an aqueous solution of a salt and a solvent, said ionic salt having a concentration in the range of 30-35.9 parts sodium chloride per 100 ml of said solution, wherein upon applying said aqueous solution to a scalp being for designated periods, said solution helps prevent loss of hair.

14. A composition of matter for preventing hair loss, said composition comprising:
an aqueous solution of sodium chloride and water, said sodium chloride having a concentration in the range of 30-35.9 parts sodium chloride per 100 ml of water, wherein upon applying said aqueous solution to a scalp of a human being for designated periods, said solution prevents loss of hair, said aqueous solution being operable to be applied from a dispenser, said scalp being at least partially covered with a polymeric cover after application of said aqueous solution, said polymeric cover being at least partially covered with a warmth generating cover for a predetermined duration.
